(19) United States
(12) Patent Application Publication
(10) Pub. No.: US 2026/0290532 A1
(43) Pub. Date: Sep. 24, 2026

(54) SYSTEM AND METHOD FOR DIFFERENTIATING MEDICATION INFORMATION TO ENHANCE USER COMPREHENSION

(71) Applicant: Express Scripts Strategic Development, Inc., St. Louis, MO (US)

(72) Inventors: Jeannien Engelbrecht, Wake Forest, NC (US); Murat Sanli, Kirkwood, MO (US)

(21) Appl. No.: 19/576,096

(22) Filed: Mar. 24, 2026

Related U.S. Application Data
(60) Provisional application No. 63/776,588, filed on Mar. 24, 2025.

(51) Int. Cl. G16H 20/00

(52) U.S. Cl. CPC G16H 20/00

(57) ABSTRACT

A medication information differentiation system includes a mobile computing device having one or more processors, one or more memory devices, an optical sensor, a display device, and an audio device. The processor(s) identify a medication by decoding a machine-readable code on a medication container. The processor(s) retrieve corresponding medication information from a database; obtain a user identifier that specifically associates a user; and determine a cognitive profile of the user based on the user identifier by accessing a user database. The processor(s) select an expression format, from among multiple different expression formats based on the cognitive profile of the user to enhance comprehension of the medication information. The processor(s) control the display device and/or the audio device to present the medication information interactively in the selected expression format. The system improves medication adherence, user engagement, and accessibility for diverse user needs.

### CROSS-REFERENCE TO RELATED APPLICATIONS

This application claims priority to U.S. Provisional Application No. 63/776,588 (filed 24 Mar. 2025), the entire disclosure of which is incorporated herein by reference.

### FIELD

The subject matter herein relates to an interactive system and method for providing medication information to a user.

### BACKGROUND

Medication for personal consumption is contained in bottles, flexible packages, and disposable applicator devices. These various forms of packages typically include labels that print information about the medication. The printed label can include dosage instructions such as the amount of the medication to ingest, the means by which to ingest the medication, and the frequency of administering the medication. The printed label can also include additional relevant information, such as restrictions/prohibitions, an expiration date, possible side effects, adverse drug interactions, contact information for the pharmacy or other healthcare provider, and the like.

Printed labels are a one-dimensional presentation of prescription insights that lack user engagement and require the user to have a standard language, communication level, and comprehension level. People have different cognitive abilities, so populations of people may not be able to read and/or understand the information printed on the label. For example, some people may not be able to comprehend the information on the label due to a vision impairment, cognitive decline associated with old age, low reading and/or education level, a learning disability, or the like. Furthermore, the labels are static. The print size is often very small to accommodate a significant amount of information in a relatively small area. Some literate users may not be able to read the label due to the small font size and/or the font style.

Some mobile applications and devices have text-to-speech technology for audibly “speaking” the content of a printed label out loud. A user can employ one of these mobile applications or devices to listen to the medication information, rather than attempt to read the printed text on the label. These mobile applications and devices provide an alternative means for allowing a user to receive and interpret the printed medication information, but the utility is limited. The known bottle reader mobile applications and devices do not differentiate how the medication information from the label is expressed to the user based on the specific user that is receiving the information. These mobile applications and devices cannot accommodate users that have different comprehension abilities, such as cognitively impaired individuals. A bottle reader application that audibly recites content of a prescription medication label would not enhance the comprehension of a hearing impaired person. The bottle reader application would also fail to support a second person that, due to a cognitive disorder, is not able to understand and/or remember spoken words. Therefore, the known bottle reader mobile applications and devices have limited effectiveness at enhancing the ability of a patient or caregiver to comprehend the medication information that is provided on the label. Furthermore, the known bottle reader applications and devices can only relay the static information that is printed on the label and/or stored in a database. Users have limited ability to interact with the known bottle reader applications and devices. For example, the applications and devices may not be able to field user-submitted questions about the medication.

When patients and/or patient caregivers are not able to reliably comprehend the medication information, the patients are less likely to comply with the intended use of the medication. Low adherence reduces the effectiveness of the medication, delaying or diminishing the therapeutic effects on the patient. Furthermore, low comprehension of the medication information may result in an increased volume of calls from patients and/or caregivers to service centers and/or healthcare providers seeking assistance.

### SUMMARY

In one or more embodiments, a computer-implemented method for dynamically providing medication information to a user can comprise one or more of the following steps: receiving, via an optical sensor of a mobile computing device, image data depicting a machine-readable code printed on a medication container label; decoding that code to identify a medication and retrieving corresponding information from a medication database; determining the container's pose in a live camera view by fusing extracted visual features with inertial measurements; obtaining a user identifier and deriving a cognitive profile by accessing a user database; acquiring device-level language and accessibility settings via operating-system accessibility APIs and reconciling them with stored user-profile preferences; selecting, based on the cognitive profile and reconciled settings, an expression format that includes an overlay anchored to the container's pose, a text complexity level, synchronized audio narration, and closed captions in the user's language; initiating a presentation mode upon successful code decoding and user authentication; registering the overlay through perspective-correct transformations to maintain anchoring under device motion; adapting overlay parameters via an adaptive readability engine in response to ambient light and motion blur; and controlling the display and audio devices to present the overlay, narration, and captions in the selected expression format.

A system for dynamically providing medication information via a mobile computing device includes a non-transitory, computer-readable memory and one or more processors operably connected to an optical sensor, a display, an audio device, and a microphone, wherein the processors are configured to: decode a machine-readable code to identify a medication; obtain medication details from a database; fuse camera and inertial data to determine container pose; retrieve a user identifier and derive a cognitive profile; access and reconcile device-level and user-profile language and accessibility preferences via operating-system APIs; select an expression format with a pose-registered overlay, text complexity control, synchronized captions, and audio narration; control the display and audio outputs with sub-frame caption synchronization; adapt overlay parameters based on illumination and motion blur; and expose accessibility metadata on graphical elements to enable screen readers and braille devices to announce interactive content.

A non-transitory computer-readable medium storing instructions that, when executed by a mobile computing device operably connected to an optical sensor, display, audio device, and microphone, cause the device to: decode a machine-readable code and determine container pose via sensor fusion; retrieve medication information and determine a user's cognitive profile; acquire and reconcile language and accessibility preferences; select an overlay-based expression format with controlled text complexity, synchronized captions, and audio narration; adapt overlay characteristics via an adaptive readability engine under varying light and motion conditions; control the display and audio outputs to render the overlay and narration with synchronized captions; and recognize voice commands to control overlay playback.

### BRIEF DESCRIPTION OF THE DRAWINGS

FIG. 1 is a block diagram illustrating a medication information differentiation (MID) system formed in accordance with one or more embodiments.

FIG. 2 illustrates a mobile computing device of the MID system according to an embodiment.

FIG. 3 is a block diagram showing information stored in the MID system and accessed by a controller to implement the MID system according to an embodiment.

FIG. 4 is a flow chart of a method for dynamically providing medication information to a user according to one or more embodiments.

FIG. 5 illustrates an example menu of module options of a graphical user interface that is displayed on a display screen of the MID system.

### DETAILED DESCRIPTION

Embodiments described herein can be directed to a medication information differentiation systems and methods that dynamically provide medication information to a user in a way that supports and enhances the user's comprehension of the information. Some embodiments address the situation of the conventional printed labels and medical literature being a one-dimensional presentation of prescription insights or medical insights that lack user engagement and require the user to have a standard language, communication level, and comprehension level. People have different cognitive abilities, so populations of people may not be able to read and/or understand the information printed on the label. For example, some people may not be able to comprehend the information on the label due to a vision impairment, cognitive decline associated with old age, low reading and/or education level, a learning disability, or the like. Furthermore, the labels are static. The print size is often very small to accommodate a significant amount of information in a relatively small area. Some literate users may not be able to read the label due to the small font size and/or the font style.

Some mobile applications and devices have text-to-speech technology for audibly “speaking” the content of a printed label out loud. A user can employ one of these mobile applications or devices to listen to the medication information, rather than attempt to read the printed text on the label. These mobile applications and devices provide an alternative means for allowing a user to receive and interpret the printed medication information, but the utility is limited. Many known bottle reader mobile applications and devices do not differentiate how the medication information from the label is expressed to the user based on the specific user that is receiving the information. These mobile applications and devices cannot accommodate users that have different comprehension abilities, such as cognitively impaired individuals. A bottle reader application that audibly recites content of a prescription medication label would not enhance the comprehension of a hearing-impaired person. The bottle reader application would also fail to support a second person that, due to a cognitive disorder, is not able to understand and/or remember spoken words. Therefore, the known bottle reader mobile applications and devices have limited effectiveness at enhancing the ability of a patient or caregiver to comprehend the medication information that is provided on the label. Furthermore, the known bottle reader applications and devices can only relay the static information that is printed on the label and/or stored in a database. Users have limited ability to interact with the known bottle reader applications and devices. For example, the applications and devices may not be able to field user-submitted questions about the medication.

When patients and/or patient caregivers are not able to reliably comprehend the medication information, the patients are less likely to comply with the intended use of the medication. Low adherence reduces the effectiveness of the medication, delaying or diminishing the therapeutic effects on the patient. Furthermore, low comprehension of the medication information may result in an increased volume of calls from patients and/or caregivers to service centers and/or healthcare providers seeking assistance.

Embodiments described herein are directed to a medication information differentiation system and method that dynamically provide medication information to a user in a way that supports and enhances the user's comprehension of the information. The medication information corresponds to a medication. The medication may be prescribed by a healthcare provider or may be available for purchase over-the-counter. The medication information differentiation (MID) system described herein differentiates the way in which the medication information is expressed (e.g., provided) to the user, based on a specific condition or ability of the user to receive and understand the medication information. The MID system is interactive, enabling the user to submit questions and provide inputs. The MID system is implemented in one or more computing devices. For example, the MID system may be an application (e.g., a software program) that operates on a mobile computing device. In an example, the mobile computing device may be a user's phone, tablet computer, laptop computer, or the like. By differentiating the way that the medication information is expressed to the user and interacting with the user, the MID system and method described herein enhance the user's comprehension (e.g., understanding) of the medication information. The user is more likely to comply (e.g., adhere) with the intended usage of the medication when the user is able to comprehend the information associated with the medication. The MID system and method may improve patient outcomes and reduce the number of calls made to service centers and/or healthcare providers.

In an embodiment, the MID system receives a first input from a user via a software application operating on a mobile computing device. The MID system identifies a medication based on the first input and obtains medication information about the medication that is identified. The MID system obtains a user identifier that uniquely identifies the user, and determines a cognitive profile of the user based on the user identifier. The MID system selects a first expression format, of multiple different expression formats, for presenting the medication information to the user based on the cognitive profile of the user. The MID system selects the first expression format to enhance comprehension of the medication information by the user, relative to selecting a different, second expression format (of the multiple different expression formats) for presenting the medication information to the user. The MID system then controls an audio device and/or a display device to present the medication information to the user according to the first expression format.

The MID system and method described herein recognize that people using the MID system, referred to herein as users, have varying language, reading, sensory, cognitive, and communication abilities. The cognitive profile of the user contains information about the condition of the user, particularly with respect to the user's ability to receive and comprehend medication information. The MID system uses the cognitive profile of the user to select the expression format at which the medication information is presented to the user, for enhancing the user's comprehension of the medication information. Comprehension in this context refers to the user's ability to receive the medication information, understand the medication information, and remember at least some of the medication information for at least a threshold amount of time (e.g., two hours, one day, or the like). The MID system chooses the way in which to convey the medication information to accommodate the specific condition of the user. As an example, the MID system may not change the baseline concepts or substance of the medication information for two different users, but may differentiate the way in which the concepts is expressed to the two users in an attempt to promote understanding by each of the two users.

The MID system may differentiate the expression of the baseline concepts by using different modes of communication, languages, phrases, terms, speeds of spoken word, visual guides, font sizes, and/or the like. For example, the MID system may change the complexity level of the medication information relative to the text of the medication information that is printed on the label. The MID system may simplify the text complexity of the printed medication information on the label or other medical literature for a first user, so that the medication information presented to the first user has more common vocabulary terms, fewer words, and/or less complex sentences than the printed label. The impetus for simplifying the text complexity may be, for example, that a first user's cognitive profile indicates that the first user has difficulty understanding the medication information on the printed label due to limited literacy skills, limited education, cognitive decline due to old age, and/or the like.

One or more technical effects of the MID system and method described herein include providing medication insights and services to patients via a convenient, customer-centered application. The MID system promotes user confidence by enabling interactive communications through computer-based input as well as voice. The MID system provides timely and accessible, on-demand medication information about a patient's medication. One technical effect is that the MID system may increase patient adherence (e.g., compliance) to the instructions provided by healthcare providers (e.g., doctors, pharmacists, medication manufacturers) for taking the medication. Increased adherence may lead to improved patient outcomes by improving the therapeutic effect of the medication on the patient, relative to the patient not complying as well with the usage instructions.

Another technical effect is the MID system and method may reduce the volume (e.g., number) of calls placed by patients and caregivers seeking assistance with the medication. For example, the MID system is interactive and may receive and provide appropriate responses to user questions using interactive voice response (IVR) technology. Furthermore, because the MID system expresses the medication information in a way that is selected based on the cognitive profile of the user, the user may better understand the medication information presented by the MID system than by attempting to read the printed label and/or listen to the text of the label read out loud by a bottle reader application. Due to the enhance comprehension of the medication information, the user would be less likely to seek assistance from a healthcare provider. A reduction in the number of calls to healthcare providers may enable the healthcare providers to increase efficiency and improve output.

The MID system demonstrates an improvement in the basic ability of a computing device to interact with users. The computing device is better able to interact with users by dynamically differentiating the way that the information is expressed to the user based on the user's specific ability to understand the selected expression format. The computing device provides the medication information to the user, in the selected expression format, by controlling a display device and/or an audio device. For example, the computing device may modify a text complexity of the medication information as printed on the label of a pill bottle, so that the display device and/or the audio device presents the medication information to the user using terms, phrases, and sentences that are likely to be received, analyzed, understood, and remembered by the user. At least some of the selected terms, phrases, and sentences may be different than the respective terms, phrases, and sentences printing on the label. The MID system provides a specific technical improvement over prior systems which cannot differentiate the way that medication information is presented to users. The problem of failure to differentiate how medication information is presented is necessarily rooted in computer technology. The known prior systems, such as bottle reader applications, have limited interactivity and are generally limited to changing the mode of communication from printed matter on the label to spoken word via a computer-generated voice or recorded human voice, without any ability to modify the expression based on specific user comprehension ability. The MID system described herein represents an improved user interface application for mobile computing devices. These and other technical benefits of the MID system and method are described in more detail herein.

The terms “user” and “users” refer to persons that administer a medication to patient, including determining when, how, and how much medication the patient should take on a day-to-day basis. The user can be the patient and/or a caregiver of the patient. Example caregivers include parents of a child patient, adult children of an adult patient, and nurses. The medication can be a prescription drug, an over-the-counter drug, or the like.

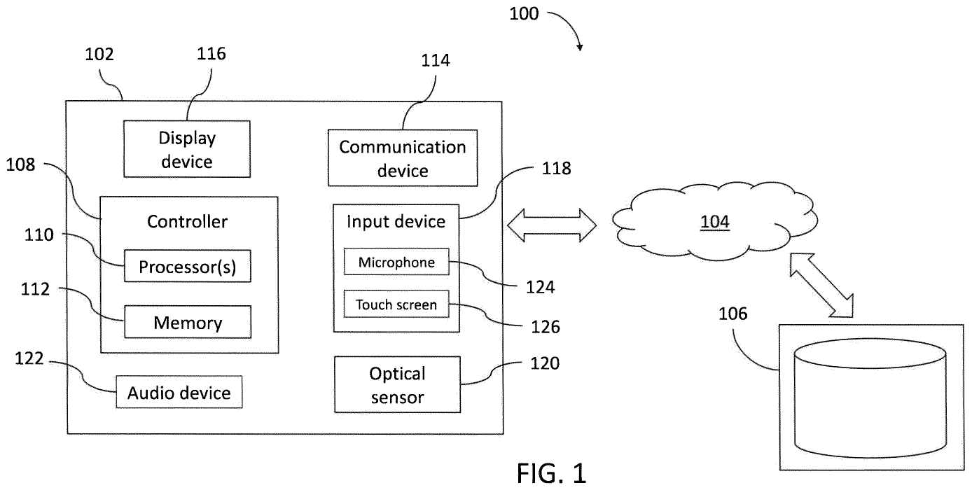

FIG. 1 is a block diagram illustrating a medication information differentiation (MID) system 100 formed in accordance with embodiments herein. The MID system 100 includes a mobile computing device 102 that is capable of communicating via a network 104 with one or more remote computing devices 106. The remote computing device(s) 106 may include one or more servers, computer towers, desktop computers, workstations, and/or the like. The remote computing device(s) 106 may be located at a remote computing facility, such as a cloud computing facility. The mobile computing device 102 may be a personal computing device owned or at least operated by a user. The mobile computing device 102 may be a phone (e.g., smartphone), tablet computer, laptop computer, or the like. In some examples, the mobile computing device 102 may be portable and/or handheld. The mobile computing device 102 performs at least some of the operations described herein to interact with a user and provide medication information to the user.

In an example, the mobile computing device 102 and the remote computing device(s) 106 may form a cloud-type computing system (e.g., public cloud, private cloud, or hybrid cloud). The network 104 may be a cable network, the Internet, a private intranet, a local area network, a wide area network, a wireless network, and/or the like. The communication pathways of the network 104 between the mobile computing device 102 and the remote computing device(s) 106 may be defined by wireless segments and/or wired segments. For example, the mobile computing device 102 may wirelessly communicate with one or more cell towers, and the cell towers may communicate through a wired connection to the remote computing device 106 at a remote cloud storage facility to complete the communication pathway.

The mobile computing device 102 includes a controller 108. The controller 108 represents hardware circuitry that includes and/or is connected with one or more processors 110 (e.g., one or more microprocessors, integrated circuits, microcontrollers, field programmable gate arrays, etc.). The controller 108 may include one or more application servers and, optionally, one or more web servers. The controller 108 includes and/or is connected with at least one tangible and non-transitory computer-readable storage medium 112 (referred to herein as memory and memory device). The memory 112 may be or include a random-access memory, a flash memory, a magnetic/optical storage, or the like. The processor(s) 110 are communicatively connected to the at least one memory 112, such as via electrical wires, electrical cables, circuit traces, fiber optics, and/or the like.

The memory 112 may store computer-readable programmed instructions (e.g., software) that are executed by the processor(s) 110 to perform at least some of the operations described herein. The programmed instructions can instruct the processor(s) 110 how to implement a software application on the mobile computing device 102 to provide medication information about a medication to a user for enhancing the user's comprehension of the medication information. For example, the programmed instructions may instruct the processor(s) 110 how to generate a graphical user interface (GUI) for the software application that is displayed on a display device. The memory 112 may be used as a repository for storing various information accessed and utilized by the processor(s) 110 when operating the software application. For example, the memory 112, or another memory device of the mobile computing device 102, may store user information about different specific users (e.g., people), a local data library that includes a record of historical activity of the software application, medication information for multiple different medications, and/or the like. The processor(s) 110 may access various portions of the memory 112 to obtain (e.g., retrieve) relevant information for providing the medication information about a specific medication to a specific user.

The mobile computing device 102 may have a communication device 114 that enables the controller 108 to send and receive information via the network 104. The communication device 114 can represent circuitry that can communicate electrical signals wirelessly and/or via wired connections. For example, the communication device 114 can include transceiving circuitry, one or more antennas, modems, routers, or the like. The transceiving circuitry may include a transceiver or separate transmitter and receiver devices. The electrical signals can form data packets that in the aggregate represent messages, such as request messages and response messages. In various embodiments, the controller 108 may use the communication device 114 to communicate with the one or more remote computing devices 106 via the network 104. The controller 108 may communicate with the remote computing devices 106 to implement and perform the operations of the MID system and method described herein. For example, the controller 108 may generate messages that are remotely communicated by the communication device 114, and the controller 108 may receive messages that are initially received and forwarded to the controller 108 by the communication device 114.

The mobile computing device 102 may include or be communicatively connected to a display device 116. The display device 116 is controlled by the controller 108 to display visual content to the user on a screen of the display device 116. The visual content includes graphical indicia, such as images, shapes, text, and symbols. In an embodiment, the display device 116 presents a graphical user interface (GUI) of a software application or website that implements the MID system 100 and method described herein. The display device 116 may be integrated into the mobile computing device 102, such as within the housing of the mobile computing device 102. Alternatively, the display device 116 may be a discrete monitor that is communicatively connected to the mobile computing device 102 via a wired or wireless connection. In an example, the visual content can be formed using cognitive fit analysis to improve understanding ot the medical information to the user of the GUI.

The mobile computing device 102 may include at least one input device 118 (also referred to herein as user input device). Each input device 118 is operable to enable the user of the mobile computing device 102 to interact with the MID system 100. The user may actuate the input device(s) 118 to submit user commands and/or selections for providing information to and/or controlling operation of the MID system 100. The input device 118 generates a control signal, in response to the user's input, which is communicated to the controller 108. The input device(s) 118 may include a touch sensitive display screen 126, a touch pad, a mouse, a keyboard, a microphone 124, a joystick, physical buttons, and/or the like. For example, the user may use one of the input devices 118 to activate or turn “on” the mobile electronic device 102, to download, launch, activate and/or turn “on” a software application or program of the MID system 100, and the like. In an embodiment, the mobile computing device 102 may include multiple user input devices 118, such as a microphone 124 for receiving user voice commands and a touch screen 126, touch pad, and/or keyboard for receiving user touch inputs.

The mobile computing device 102 may include at least one optical sensor 120. The optical sensor(s) 120 may include a camera, an electromagnetic scanner or reader device, and/or the like. For example, the optical sensor 120 may be a camera that captures (e.g., generates) image data based on light in the surrounding environment that enters a lens and impinges upon a digital sensor of the camera. In another example, the optical sensor 120 may be an electromagnetic scanner or reader that scans a code printed on a tangible item, such as a barcode, a quick-response (QR) code, or a radio frequency identification (RFID) tag on a medication container. The medication container could be a pill bottle, a flexible package, a box, a single use applicator device, or the like.

The mobile computing device 102 may include at least one audio device 122. The audio device(s) 122 may be a speaker with an amplifier to emit sounds based on electrical signals. The controller 108 may generate the electrical signals that are communicated to the audio device(s) 122 and output as audio. As an example, the controller 108 may control the audio device(s) 122 to emit machine-generated speech and/or human-recorded speech. The audible speech that is emitted from the audio device(s) 122 may describe the medication information for the specific medication.

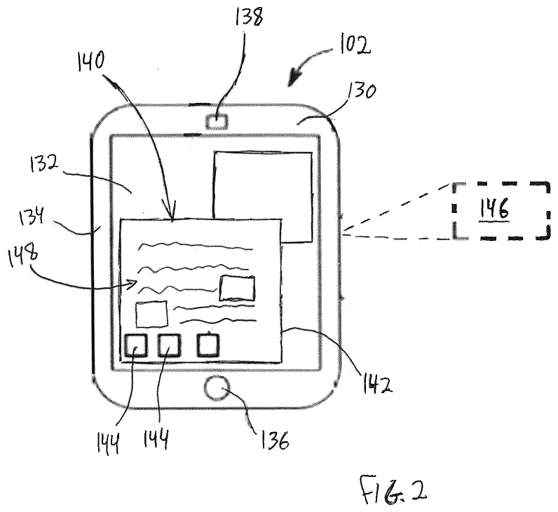

FIG. 2 illustrates the mobile computing device 102 of the MID system 100 according to an embodiment. The mobile computing device 102 in FIG. 2 is illustrated as a tablet computer. For example, the mobile computing device 102 includes a housing 130 that is relatively flat and planar. A display screen 132 of the display device 116 (shown in FIG. 1) occupies a majority of the front, or user-facing, side 134 of the mobile computing device 102. The housing 130 may frame the display screen 132. The display screen 132 may be touch-sensitive, to also function as one input device 118 (shown in FIG. 1). The mobile computing device 102 may also have additional input devices 118, such as a user-selectable button 136 next to the display screen 132. The mobile computing device 102 may include a camera 138 that represents the optical sensor 120 (shown in FIG. 1). The user may employ the camera 138 for optically scanning or imaging a medication container for identifying the medication. The tablet-style mobile computing device 102 shown in FIG. 2 is merely an example computing device on which the MID system 100 could be implemented. The mobile computing device 102 may have other form factors, shapes, sizes, and the like in other examples. For example, the MID system 100 could be implemented on various different types of computing devices, including different types of mobile computing devices 102.

The controller 108 (shown in FIG. 1) of the mobile computing device 102 may control the display device 116 to present a graphical user interface (GUI) 140 for the MID system 100 on the display screen 132. The GUI 140 manages the interaction between the MID system 100 and the user through graphical elements, such as windows 142 and user-selectable input elements 144. The windows 142 display various types of outputs for various computer processes corresponding to the MID system 100. Multiple windows 142 may be concurrently displayed. The GUI 140 may present a visual guide 148 for understanding the medication information. The visual guide 148 may include images, symbols, text, illustrations, videos, interactive games, or the like.

The user-selectable input elements 144 (referred to herein as input elements) accept user input for controlling the computer processes of the MID system 100. The input elements 144 may be virtual buttons that are selectable via a touch input on the area of the display screen 132 showing the input element 144 or via a user-movable cursor that is displayed on the screen 132. In an example, a first input element 144 may be selectable by the user to initiate a call with a healthcare provider, such as a pharmacy or doctor's office. A second input element 144 may be selectable by the user to enable the user to submit an inquiry or command, such as a voice command. A third input element 144 may be selectable to allow the user to request a refill of the prescription medication. The GUI 140 may have any number of input elements 144 for allowing the user to interact with the MID system 100.

In an embodiment, the MID system 100 voice compatibility may enable a user to select desired input elements 144 via voice commands, without requiring touch inputs. For example, a user may press a button or state out loud a trigger term to activate the microphone 124. With the microphone 124 active, the user may then say one of the input elements 144 to select that corresponding input element 144. Upon receiving the voice command, the controller 108 may modify the GUI 140 to open a new window, text box, or the like and present additional information specific to that selected input element 144. As an example, if the user asks out loud, “what are the potential side effects?” the controller 108 may modify the GUI 140 to present information about potential side effects of the specific medication to the user. Concurrently with the presentation of the modified GUI 140, the controller 108 may control the audio device(s) 122 to emit computer-generated or human-recorded speech reciting information about the potential side effects of the specific medication.

The MID system 100 may include a software application 146 that operates on the mobile computing device 102. The software application 146 is a program that may be stored locally on the mobile computing device 102, such as in the memory 112 or another local non-transitory computer-readable storage medium. The software application 146 permits the user to interact with the MID system 100. For example, the software application 146 may generate the GUI 140 that displays graphical content to the user and receives user input. The controller 108 may host the software application 146, including the tools, application program interfaces (APIs), and scripts, among other things, that may be used for the software application 146. Optionally, a user may download the software application 146 for the MID system 100 onto their mobile computing device 102. The user may open, launch, or initiate the software application 146 to access and implement the MID system 100. As an example, the user may open the software application 146 by selecting an icon that represents the software application 146. The software application 146 may require at least one form of authentication from the user before granting the user access to a personal account. Accepted forms of authorization may include a password, a security code on a trusted device, face or other biometric recognition, and/or the like.

In an example, upon downloading the software application 146, the software application 146 may determine (e.g., access or obtain) desired language settings for the user. The desired language settings may include various languages, such as English, Spanish, French, sign languages such as American Sign Language, and/or the like. The language settings may also include braille and other forms in which the languages may be expressed or provided to the user. For example, the MID system 100 (and software application 146 thereof) may be compatible with external braille display devices, may contain a braille-type mobile screen feature, and/or the like. Some computing devices may be used by various different users over time. In an example, to accommodate multiple different users with varying language settings, the software application 146 may access or obtain the user's language settings once the user successfully logs into the software application 146. For example, upon authenticating that the user has a stored profile, the controller 108 may access and retrieve all settings specific to that user, including language settings. The MID system 100 may use the user's language settings to present the medication information to the user and interact with the user. The user language settings and other user-specific settings may be selectable and modifiable in a settings menu of the software application 146.

The MID system 100 can obtain the preferred language settings directly from the operating system of the mobile electronic device 108 prior to launch of the AR mode, and can reconcile such device-level preferences with language settings stored in the user's profile within the user database 204. The controller 108 can read local, language, and accessibility preferences exposed by the operating system and determine final presentation settings for the session. If a conflict exists between device-level language settings and user-specific stored preferences, the controller 108 may apply a predetermined rule set that prioritizes user profile settings or prompts the user via the GUI 140 to select a preference. The selected language setting can be used to stage all subsequent experiences, including AR overlays, captions, IVR prompts delivered via the audio device 122, and GUI modules displayed on the display device 116, so that the medication information 210 is presented in the chosen language throughout the session. This rule set can be part of a cognitive fit engine or AI to clarify the medical timing, reduce information overload, improve at least one of spatial reasoning and symbolic reasoning, and safety-critical outcomes.

The software application 146 may be configured to work with sub-applications or scripts (e.g., plug-ins or extensions) that are executed from within the software application 146 or in concert with the software application 146. The software application 146 may retrieve and communicate information through the network 104 (shown in FIG. 1), such as by communicating with the remote computing device(s) 106. The software application 146 may communicate using, for example, a known protocol (e.g., Hypertext Transfer Protocol (HTTP) or HTTP-secure (HTTPS)). More specifically, the software application 146 may send requests (e.g., HTTP requests) for information to any web-accessible internet address. The software application 146 may display information on the mobile computing device 102 in accordance with a predetermined format (e.g., HTML format). Embodiments set forth herein may be implemented, at least in part, using the software application 146, a sub-application associated with the software application 146, or other software program having computer executable code.

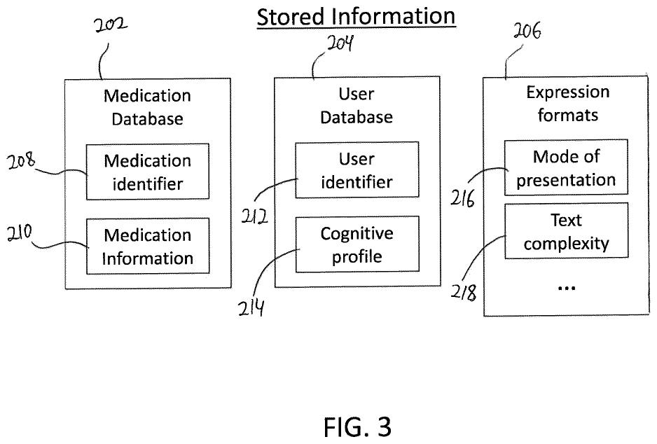

FIG. 3 is a block diagram showing categories of information stored in the MID system 100 and accessed by the controller 108 to implement the MID system 100 according to an embodiment. The information is grouped into a medication database 202, a user database 204, and expression formats 206. This information identified in FIG. 3 can be stored locally in the memory 112 of the mobile computing device 102 and/or remotely in the one or more remote computing devices 106. Portions of the information remotely stored are accessible to the controller 108 via the network 104. Portions of the information locally stored are accessible to the processor(s) 110 via circuitry within the mobile computing device 102. The boxes in FIG. 3 are meant to identify groups (e.g., categories, classes, etc.) of related information, rather than denote physical boundaries or indicate that the information is stored in different discrete memory devices. For example, all of the information identified in FIG. 3 could be stored in a single remote computing device 106.

The medication database 202 includes stored medication identifiers 208 and medication information 210. The stored medication identifiers 208 are used by the MID system 100 to identify a particular medication. The stored medication identifiers 208 may include specific alphanumeric characters, barcodes, QR codes, RFID tags, electrical signals generated by the optical sensor 120 in response to scanning a medication package, and/or the like. The stored medication identifiers 208 may be stored in a list so that each identifier is associated with a specific medication. The controller 108 may receive a medication identifier as a first input. The medication identifier may be provided on a physical medication package in possession of the user that is using the MID system 100. For example, the user may view the medication identifier on the package and then use an input device 118 to input the medication identifier into the MID system 100. In another example, the medication identifier may be input to the MID system 100 by using the optical sensor 120 to scan the package. The controller 108 may detect and/or determine the medication identifier by the user moving the mobile computing device 102 so that the field of view of the optical sensor 120 encompasses a barcode, QR code, text, or the like of the medication package. Upon determining a medication identifier on the package, the controller 108 may then access the stored medication identifiers 208 to identify the medication possessed by the user.

In an example, the controller 108 may receive the first input from the optical sensor 120. For example, the optical sensor 120 may optically scan a machine-readable code printed on a label of the medication container. The machine-readable code may be a barcode, a QR code, or the like. The optical sensor 120 in another example may capture image data of alphanumeric characters printed on the label, and the controller 108 may analyze the image data and optically recognize the characters to determine the medication identifier. The optical sensor 120 may alternatively be an RFID reader device that electromagnetically scans an RFID tag coupled to or embedded within the medication container. In another example, the controller 108 may receive the first input from the user input device 118. For example, the user may read one or more identifying words printed on the medication container, such as the name of the medication, and may use the GUI 140 on the mobile computing device 102 to input text characters spelling the one or more identifying words from the container.

The medication information 210 refers to information that is relevant to, and instructions for, administering the particular medication associated with the stored medication identifier 208 that is determined. The medication information 210 can include usage instructions, basic descriptive information about the medication, restrictions and/or prohibitions, drug interactions, possible side effects to taking the medication, resources, and the like. The source of the medication information 210 may be one or more doctors, pharmacists, medication manufacturers, and/or the like. The usage instructions may indicate the means or way in which the user is to administer the medication, the amount of the medication to administer each dose, the frequency of the doses, and/or the like.

The basic information about the medication can include the common name of the medication, a scientific name for the medication, a shape of a unit (e.g., pill) of the medication, a unit color, a unit size, ingredients of the medication, the manufacturer of the medication, the pharmacy that provided the medication in the medication container, the date at which the medication was manufactured, an expiration date for the medication, intended positive effects (e.g., benefits) of taking the medication, and/or the like. The restrictions and/or prohibitions indicate one or more reasons, scenarios, or conditions in which the patient should not take the medication. Example restrictions and/or prohibitions can include warning the patient not to take the medication if the patient is pregnant or breastfeeding, warning the patient not to take the medication with alcohol, and/or the like. The drug interactions identify other medications that may not combine well with the medication in the patient's system. The possible side effects identify common side effects of other patients that have taken the medication in the past. The resources provide information for finding additional information. For example, the resources may include contact information for a pharmacy, a doctor's office, or other healthcare provider.

The controller 108 receives the first input and uses the medication identifier of the first input to identify the particular medication and the medication information 210 associated with the identifier. For example, the medication information 210 may be stored in a database with the stored medication identifiers 208. As such, when the controller 108 determines that the first input indicates a stored medication identifier “A”, the controller 108 accesses the database to retrieve the medication information 210 that corresponds to the medication associated with identifier “A.” Optionally, the medication database 202 may be an ordered list, a table, or the like that provides corresponding stored identifiers 208 and medication information 210 for a multitude of different medications.

The user database 204 includes user identifiers 212 and cognitive profiles 214. The user identifiers 212 uniquely identify users that operate the MID system 100. For example, user identifiers 212 may be user credentials that are stored in a database, where each user credential is associated with a different user. The controller 108 identifies the user so that the controller 108 can determine the cognitive profile 214 of the specific user, which is used to determine the expression format for presenting the medication identification to the user in a way that enhances the user's comprehension of the medication information. The user database 204 may include an ordered list, table, or the like that includes the user identifiers 212, user identifications (e.g., names), and the cognitive profiles 214 corresponding to the user.

As an example, a first user named John may open or launch the software application 146 for the MID system 100 on the mobile computing device 102. The GUI 140 may prompt John to input, via an input device 118, user credentials associated with an account. Assuming John has an account, John would enter his user credentials, such as a user name and password, a temporary code sent to an external, linked email address, or the like. Optionally, the software application and mobile computing device 102 may enable biometric authentication, such as scanning John's eye or fingerprint. The input provided by the user. such as biometric data, user credentials, or the like, is then compared by the controller 108 to the stored user identifiers 212 to determine a match. If there is a match, then the user is positively identified. Once the user is positively identified (e.g., the user's identity is known), the controller 108 can obtain the cognitive profile 214 of the user because the user identifiers 212 are associated with different corresponding cognitive profiles 214. The controller 108 may also obtain the user-specific settings that are saved for John's account, such as language settings.

In another example, John may not need to separately enter user credentials or provide biometric data if the mobile computing device 102 is uniquely associated with John. For example, John may be required to provide a passcode or biometric identification in order to access and operate the mobile computing device 102. In that case, the controller 108 may receive the user identifier 212 uniquely associated with John, from the mobile computing device 102 in response to John launching the software application 146 of the MID system 100.

The cognitive profile 214 provides information about the specific, corresponding user's ability to receive external information via the senses, analyze (e.g., process) the external information in the mind, understand the information, and remember the information. The cognitive profile 214 can be generated by a cognitive fit engine that assigns types of communication based on the cognitive score of the user. The external information that is provided to the user by the MID system 100 is the medication information 210. As such, the cognitive profile 214 describes the expected or predicted ability of the specific user to receive, understand, and remember the medication information 210. The cognitive profile 214 may include physical, mental, and/or developmental conditions, as well as educational limitations, that impair, interfere with, or limit the user's ability to perceive, interpret, comprehend, and remember the medication information from a label on a bottle, package, or printed sheet. Different users have different cognitive profiles 214.

The cognitive profiles 214 may identify specific diagnosed impairments, impediments, and/or disabilities of the users. Some examples of impairments include hearing impairment, vision impairment, learning disability, reading disability (e.g., dyslexia), attention deficit disorder, age-related cognitive decline, Alzheimer's disease, dementia, illiteracy, lack of or limited education attained, and/or the like. Optionally, the cognitive profile 214 of a user may indicate properties of the user, for example the user's level of education achieved (e.g., 8^{th} grade, high school graduation, college, grad school, etc.), reading level, vision acuity, hearing acuity, mental acumen, age, natural language, and/or the like. Some of the properties of the user in the cognitive profile 214 may be provided with a value on a scale, which enables the user to be categorized and compared to other users. As an example, the cognitive profile may include a value of 1 if the user did not attend high school, a value of 2 if the user attended high school but did not graduate, a value of 3 if the user graduated high school, and a value of 4 if the user attended college. Similarly, the cognitive profiles 214 may indicate different levels or grades of other conditions using values on a scale, including levels of hearing ability, levels of vision acuity, levels of memory retention, the user's reading level, and the like.

The MID system 100 may retain the cognitive profiles 214 as confidential information. For example, the MID system 100 may only provide information from a first cognitive profile 214, upon request, to a verified user that is associated with the first cognitive profile 214. A second user operating the MID system 100 would not be able to access the first user's cognitive profile 214. Each user may have the ability, through the GUI 140 and settings of the software application 146 of the MID system 100, to input information about that user's comprehension abilities or impairments, which is added to that cognitive profile 214.

The cognitive profiles 214 are used by the controller 108 of the MID system 100 to select an expression format of multiple different expression formats 206 that are stored. The expression formats 206 include modes of presentation 216, text complexity 218, and the like. Expression formats 206 are the framework in which the controller 108 describes and presents the medication information 210 to the user and provides resources for appropriate interpretation and understanding of the medication information 210 by the user. An expression format 206 can encompass a mode of presentation (e.g., mode of communication) 216, a form of the information communicated, a text complexity 218 at which the information is communicated, and/or the like. The mode of presentation 216 refers to the medium by which the information is conveyed by the MID system 100 to the user. Example modes include aural via the audio device 122 and visual via the display device 116. The form of the information communicated represents the elements used to convey the information, such as alphanumeric characters, spoken words, symbols, videos, games, and/or the like. The text complexity 218 refers to the complexity of the words and ideas communicated to the user. For example, text complexity 218 can include vocabulary, sentence structure, and concept sophistication of the way in which the controller 108 expressed the medication information 210 to the user. Other examples aspects of the expression format 206 can include text size, font style, speed at which the information is output (e.g., speed of spoken words), volume at which the information output, and/or the like.

The cognitive profile can be created by mapping cognitive ability to functional ability. Data stored within the medical history of a patient, including the written correspondence and recorded telephone interactions are processed and then linked to a map of a map of one or more cognitive processes underlying the cognitive ability to a continuous-valued measure of the functional ability. The output from the map map can be stored on a computer-storage medium to be used by the system to adjust the prescription label or medical literature.

The controller 108 may differentiate the expression format 206 for different users based on the users'specific cognitive profiles. One way that the controller 108 can differentiate the expression format 206 is by selecting different text complexity 218 for different users. In an example, the MID system 100 can select different discrete levels or grades of text complexity 218 from low complexity to high complexity. For example, a low or lower complexity level may have relatively short words, may convey relatively simple ideas, may supplement words with symbols, images, and/or video, and may use prose that follows the user's natural rhythm of speech and ordinary grammatical structures. A high or higher complexity level would use longer words, more extensive vocabulary terms, less symbols, images, and/or video, and would convey more complex ideas.

In an example, the MID system 100 can include an augmented reality (AR) presentation mode that is automatically initiated in response to the controller 108 identifying a medication by scanning a machine-readable code on a medication container. The mobile computing device 102 can be a portable or handheld computing platform equipped with the display device 116, the optical sensor 120 (e.g., camera), the microphone 124, and/or the communication device 114, and operable to execute the software application 146 and the controller 108. Examples include smartphones and tablet computers running mobile operating systems, smartwatches with integrated cameras or paired camera accessories capable of relaying image data to the controller 108, handheld augmented reality devices and smart glasses with see-through or video-pass-through displays, and portable laptops or convertible notebooks with integrated webcams and touch screens. In some examples, the mobile computing device 102 may be a dedicated handheld scanner with an embedded camera and display configured to run the AR modules, or a mixed reality headset that renders AR overlays in a stereoscopic display. The mobile computing device 102 may further support external peripherals, such as braille displays, Bluetooth headsets for audio output via the audio device 122, or stabilized camera rigs to improve AR registration, and may include hardware acceleration for graphics processing to render three-dimensional animations and high-contrast overlays in real time.

The machine-readable code may include a 2D barcode printed on a prescription label. Upon detecting the code via the optical sensor 120 and identifying the corresponding medication, the controller 108 transitions the software application 146 into an AR mode in which medication information 210 overlays a live camera view presented on the display device 116 of the mobile computing device 102 and surrounding environment. The AR mode presents contextual visual guides 148, including icons, simplified text, pictograms, and optionally three-dimensional animations, aligned to the container's pose so that the user can observe medication-specific insights superimposed on the physical item.

The AR mode may present medication information 210 via layered overlays that are differentiated according to the cognitive profile 214 of the user. For users with lower reading levels or documented cognitive or learning disabilities, the controller 108 selects overlays with simplified text complexity 218, larger font sizes, slower-paced animations, and enhanced iconography. For users with visual impairments, the controller 108 reduces reliance on small text, increases contrast ratios of overlays rendered on the display device 116, and emphasizes audio narration synchronized to the AR content via the audio device 122. For hearing-impaired users, the controller 108 renders closed captions for all audio prompts and provides visual emphasis effects on critical instructions such as dosage amount and timing.

The controller 108 may authenticate the user prior to launching the AR mode by requiring password, security code, or biometric verification provided by the mobile computing device 102. In one example, the controller 108 invokes the device's native authentication framework to verify the user and, upon successful authentication, enables an AR/IVR session that is triggered by the 2D barcode scan captured by the optical sensor 120. The controller 108 may also require a second factor associated with the user's account stored in the user database 204, such as a one-time code delivered to a trusted device, to ensure that access to medication information 210 is restricted to verified users.

The MID system 100 can integrate with accessibility services of the mobile operating system of the mobile electronic device 108, such as screen reader technologies such as TalkBack™ and VoiceOver™. The controller 108 can annotate all or at least some GUI 140 components and AR overlay elements with accessibility labels, roles, and hint text so that the screen reader can announce interactive controls and content to the user via the audio device 122. The controller 108 also can ensure that the AR overlays and GUI elements are navigable via focus order, keyboard access where applicable, and braille display compatibility. The controller 108 can output captions and audio transcripts for IVR prompts and AR narration and exposes toggles in the settings presented on the display device 116 for enabling captions, adjusting caption size, and selecting caption language.

The MID system 100 may include a braille-type mobile screen feature and compatibility with external braille display devices so that essential medication information 210 is accessible to users with severe vision loss. In the AR mode, when braille display compatibility is enabled, the controller 108 maps key overlay content, such as medication name, dosage instructions, contraindications, and side effects, to braille output through the operating system's accessibility APIs. The controller 108 can update the braille output in real time as the user navigates through AR overlays via the input device 118, selects modules, or requests definitions of unfamiliar terms.

The controller 108 may manage AR assets, including images, vector graphics, 3D animations, caption tracks, and localized text strings, via a content library stored in one or more memory devices 112, 106. In one example, AR assets are organized by medication identifier 208 and include variants for multiple text complexity levels 218, multiple languages, and multiple accessibility profiles. The controller 108 retrieves the appropriate assets at session start, and can cache the assets locally in the memory 112 to reduce latency during AR presentation on the display device 116. The controller 108 may prefetch assets for commonly used modules 402, such as “Your Medication,” “Drug Interactions,” and “Potential Side Effects,” based on historical usage recorded in a local data library stored in the memory 112.

The remote computing device 106 can remotely store AR assets and IVR prompts in a canonical format and provide versioned updates to the mobile computing device 102 via the communication device 114 over the network 104. The controller 108 can synchronize asset packages and converts or transcodes any session-specific updates to formats supported by the device hardware and operating system. High-resolution 3D animations can be transcoded to device-appropriate meshes or sprite sequences and caption files are converted to platform-supported caption formats. The controller 108 can maintain integrity checks and version metadata so that the AR overlays and IVR prompts presented during a session correspond to the latest approved content for the identified medication information 210.

The MID system 100 may provide an interactive voice response experience that operates concurrently with the AR overlays. As the user views AR content, the microphone 124 may receive voice commands that control playback and navigation of AR modules 402. The controller 108 recognizes voice commands including “Rewind,” “Fast forward,” “Replay,” “Stop,” and “Pause,” and adjusts AR animation timelines and audio narration emitted by the audio device 122 accordingly. The controller 108 may also support commands such as “Volume up,” “Volume down,” “Slow,” and “Speed up,” to modify narration volume and playback speed. In one example, the cognitive profile 214 determines default speed and volume settings, and the user may override those defaults with voice commands or touch inputs received via the touch screen 126.

The MID system 100 can include a term definition function within AR mode so that the user can request clarification of unfamiliar medical vocabulary encountered in overlays or narration. The controller 108 may receive a voice command “Define [word]” via the microphone 124 or a touch gesture on the touch screen 126 that highlights a term displayed on the overlay. In response, the controller 108 retrieves a simplified definition aligned to the user's text complexity selection 218 and renders the definition as a visual callout on the AR overlay displayed on the display device 116. The controller 108 may simultaneously emit audio narration of the definition via the audio device 122 and display captioned text, both of which are localized to the user's selected language.

The AR overlays may incorporate interactive modules 402 that users can select via touch on the touch screen 126, braille controls, or voice commands captured by the microphone 124. When a module 402 is selected within AR mode, such as “Auto Refill” or “Script Renewal Request,” the controller 108 may collapse or reposition AR overlays to reveal module-specific GUI panels 142 while preserving the contextual link to the medication container in view. The controller 108 may then initiate network transactions via the communication device 114 to process refills or renewal requests and display status updates as anchored overlays near the container so that the information remains contextually associated with the medication identified by the medication identifier 208.

The controller 108 may analyze the environmental conditions of the mobile electronic device 108 and adjust AR presentation parameters to maintain readability and comprehension. In low-light conditions detected via the optical sensor 120, the controller 108 can increase overlay brightness and contrast on the display device 116. In high-motion conditions where device stability is reduced, the controller 108 can reduce complex animations and pins critical instruction overlays to maintain legibility. In examples where the cognitive profile 214 indicates diminished working memory, the controller 108 can extend the dwell time of each overlay, reduces the number of concurrent elements on screen, and/or insert spaced repetition prompts that revisit essential instructions such as dosage frequency.

The MID system 100 may record anonymized interaction metrics to the local data library stored in the memory 112, such as AR modules 402 that are selected by the user, voice command usage via the microphone 124, playback adjustments of the audio device 122, and time-on-screen for each overlay rendered on the display device 116, and may synchronize aggregated metrics to the remote computing device 106 for continuous improvement of assets via the communication device 114 over the network 104. The controller 108 may use these metrics to adapt future sessions for the same user by preferring overlays and narration styles that historically correlated with longer engagement and fewer clarification requests. The controller 108 may also update recommended text complexity levels 218 for the user based on observed command usage and comprehension signals, such as the frequency of “Define” requests or repeated playback of certain instruction segments.

The controller 108 can enforce accessibility compliance across GUI 140 and AR modes by ensuring that all interactive elements expose accessibility metadata and respond to operating system-level assistive technologies, including TalkBack™ and VoiceOver™. The controller 108 may validate overlay contrast, caption timing, and focus navigation for screen readers and braille devices prior to displaying each module 402. The controller 108 may also provide an accessibility diagnostic panel in settings displayed on the display device 116 where users can test TalkBack™ or VoiceOver™ readouts via the audio device 122, adjust caption size, enable high-contrast overlays, and preview voice command recognition quality via the microphone 124. These diagnostic features may be adapted based on the cognitive profile 214 to simplify options and provide guided recommendations for users with documented impairments.

The above-described AR and accessibility embodiments may be implemented in conjunction with the previously described modules 402, databases 202, 204, expression formats 206, and communication devices 114. The AR mode, device language auto-recognition, accessibility integrations, and concurrent IVR control provide an enhanced user interface application that differentiates the presentation of medication information 210 based on the user's specific ability to perceive, process, and retain the information. By aligning AR overlays, narration via the audio device 122, captions displayed on the display device 116, and command vocabularies received through the microphone 124 to cognitive and accessibility profiles 214, the MID system 100 increases comprehension and adherence while maintaining compatibility with a range of device capabilities and assistive technologies.

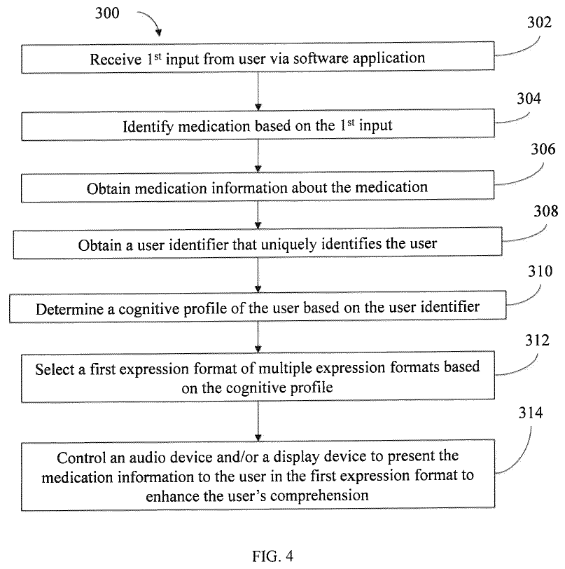

FIG. 4 is a flow chart 300 of a method for dynamically providing medication information to a user according to one or more embodiments. The method is a computer-implemented process that uses one or more non-transitory, computer-readable memory devices and one or more processors of a mobile computing device operably connected to the memory device(s). The method may be performed, at least in part, by the controller 108 (e.g., the processor(s) 110) of the MID system 100. The memory devices may include the memory 112 and/or the remote computing device(s) 106, such as at least one server. The method may include additional steps than shown in FIG. 4, fewer steps than shown in FIG. 4, a different order of the steps, and/or different steps than shown in FIG. 4.

With additional reference to FIGS. 1 through 3, the method begins at step 302, at which a first input is received from a user via a software application 146 operating on a mobile computing device 102. The first input may be received by the controller 108 by the user (i) optically scanning a machine-readable code printed on a label of a medication container; (ii) optically recognizing alphanumeric characters printed on the label, or (iii) electromagnetically scanning a radio frequency identification (RFID) tag coupled to or embedded within the medication container. For example, the user may position the label, code, and/or RFID tag in the field of view of an optical sensor 120. The optical sensor 120 may be a camera, an RFID reader, or another type of electromagnetic scanner device. The controller 108 analyzes the first input that is received to identify the medication that is within the medication container (e.g., bottle, package, single use applicator, or the like). In an example, the controller 108 may store the first input received from the user, which identifies the medication, in a first format in one or more network-based non-transitory storage devices (e.g., memories). The one or more memories have a collection of medication data stored thereon.

At step 304, the medication is identified based on the first input. The medication may be identified by accessing a first database (e.g., the medication database 202) stored in a first memory device. The first memory device may be the memory 112 or a remote computing device 106. The first database contains a list of multiple types of medication and associated medication identifiers 208. At step 306, medication information 210 about the identified medication is obtained. The controller 108 may obtain the medication information 210 about the medication by accessing the first database or another database. For example, the medication database 202 may include a list, table, or other repository that links different medications with respective associated medication information 210. After identifying the medication and/or the medication identifier 208, the controller 108 may use that information to retrieve the corresponding medication information 210. The medication information 210 is relevant information to assist the user with administering the medication.

In one example, the method can include, after step 306, automatically launching an AR presentation mode upon identifying the medication via the machine-readable code and authenticating the user. The controller 108 can select AR assets and localized text strings based on the cognitive profile 214 and preferred language settings obtained prior to launch. The controller 108 can control the display device 116 to render AR overlays that visually register to the medication container and controls the audio device 122 to emit synchronized narration with captions enabled according to accessibility preferences. The controller 108 can concurrently listen for voice commands through the microphone 124 and adjusts AR playback and narration in real time to create an interactive experience tailored to the user.

At step 308, a user identifier 212 is obtained that uniquely identifies the user that is operating the MID system 100 via the mobile computing device 102. In one example, the controller 108 may obtain the user identifier 212 by receiving user credentials, via a user input device 118, and comparing the user credentials that are received to a user database 204. For example, the software application 146 may provide a GUI 140 that prompts the user to input user credentials before the user is allowed to receive the medication information 210. The user may input the user credentials using a touch screen 126, a keyboard, a touch pad, a microphone 124, or the like. The user database 204 may contain a list of plural user identifiers and associated user credentials. The controller 108 may determine if the received user credentials match any of the user identifiers 212 in the user database 204 to authenticate the user. If the credentials match, then the controller 108 authenticates the user as the user matching a corresponding user identifier 212.

At step 310, a cognitive profile 214 of the user is determined based on the user identifier 212. The cognitive profile 214 may be determined by accessing a second database (e.g., the user database 204) stored in the first memory device or a second memory device. For example, the user database 204 may be stored locally in the memory 112 or remotely in the remote computing device 106. The cognitive profile 214 provides information about the particular user's ability to comprehend the medication information 210 of the medication. More specifically, the cognitive profile 214 may identify and/or assign values to characteristics or properties of the user that may affect the user's ability to perceive, analyze (e.g., process), understand, and remember the medication information 210.

At step 312, a first expression format 206, of multiple different expression formats 206, is selected for presenting the medication information 210 to the user based on the cognitive profile 214 of the user. The controller 108 selects the first expression format 206 to enhance comprehension of the medication information 210 by the user. The various available expression formats 206 may be stored in a memory device, such as the memory 112 or the remote computing device 106. The expression formats 206 can include modes of presentation 216, text complexity 218, and/or the like. The first expression format 206 is the framework, selected by the controller 108, in which the controller 108 will describe and present (e.g., express) the content of the medication information 210 to the user. The first expression format 206 may provide resources for appropriate interpretation and understanding of the medication information 210 by the user. The resources can include visual guides, the ability for the user to submit questions, the option to contact a third party healthcare provider, and/or the like.

At step 314, an audio device 122 and/or a display device 116 is controlled to present the medication information 210 to the user in the selected first expression format 206. The controller 108 may present the medication information 210 to the user via the audio device 122 alone, the display device 116 alone (e.g., on the GUI 140), or via both the audio device 122 and the display device 116 in combination.

By selecting the first expression format 206 based on the cognitive profile 214 of the user, the controller 108 differentiates the way that the content of the medication information 210 is expressed to the user for the purpose of enhancing the user's comprehension and retention of the medication information 210, at least relative to the user simply reading the label of the medication container and/or the audio device 122 simply reading the text of the label out loud. For example, when the first expression format 206 includes the use of the audio device 122 alone, the controller 108 may control the audio device 122 to speak at least some different words, phrases, and/or sentences than the words, phrases, and sentences printed on a label or other document provided with the medication. In another example, based on the cognitive profile 214, the controller 108 may control the audio device 122 to simply read a label out loud to the user, and may also control the display device 116 to display a visual guide 148 on the GUI 140 to enhance the user's comprehension. The visual guide 148 may include one or more images, symbols. illustrations, videos, interactive games, and/or the like related to the medication. The combination of the visual aid with the audible recitation of the printed text on the label may enhance the user's retention of the subject matter.

In at least one example, the controller 108 may control the display device 116 to display a GUI 140 on a screen 132 of the display device 116. The controller 108 may visually present at least some of the medication information 210 on the GUI 140. For example, in response to the cognitive profile 214 of the user indicating that the user has a hearing impairment, the controller 108 may control the display device 116 to visually present the medication information 210 on the GUI 140. The hearing-impaired user in this example would be more likely to perceive the information 210 using the sense of sight rather than hearing.

Optionally, the controller 108 generates the GUI 140 to include at least one input element 144, which can be selected by the user via a user input device 118. In an example, in response to receiving an input signal from the user input device 118 indicating selection of a first input element 144, the controller 108 may initiate a call with a healthcare provider. In another example, in response the user selecting a second input element 144, the controller 108 may generate a refill request for additional doses of the medication. The controller 108 may then communicate the refill request to a healthcare provider, such as a doctor's office or pharmacy. The controller 108 may use the communication device 114 to transmit the refill request in a message to the healthcare provider.

In another example, in response to the cognitive profile 214 of the user indicating that the user has a vision impairment and/or a reading impairment, the controller 108 selects the first expression format 206 that includes controlling the audio device 122 to audibly present the medication information 210 to the user. For example, if the user is not able to reliably read the printed information, then the controller 108 can assist by speech. The first expression format 206 may include characteristics such as the speed and/or volume at which the audio device 122 recites the medication information 210. As part of selecting the first expression format 206, the controller 108 may select a first speed of multiple different speeds and/or a first volume level of multiple different volume levels. As an example, if the cognitive profile 214 indicates that the user has diminished cognitive ability due to old age, the first volume level may be louder and the first speed may be slower than if the cognitive profile 214 indicates that the user does not have a diminished cognitive ability.

As an example, if the cognitive profile 214 indicates that the user has the reading impairment, the controller 108 may select the first expression format 206 to include both the audio device 122 audibly presenting the medication information 210 to the user and the display device 116 presenting the visual guide 148 on the GUI 140 to assist understanding the medication information 210. The combination of the visualizations with the voice recitation may increase the user's retention of the medication information 210.

In an example, the controller 108 may select the first expression format 206 by, in part, selecting a level of text complexity 218 (e.g., text complexity level) based on the cognitive profile 214 of the user. The text complexity level refers to length of words that are provided to the user, prevalence of the words in ordinary use as experienced by the user, and length and complexity of sentences that are provided to the user. The level of text complexity 218 may be selected based on the reading level of the user, the education level of the user, and/or the presence or absence of any cognitive disabilities. For example, the controller 108 may select a higher text complexity level for a first user that has graduated college and has no documented cognitive disability than for a second user that has less education and/or a lower reading level than the first user and/or has a documented cognitive disability that would interfere with understanding more complex terms and sentence structures. The controller 108 may select the first expression format 206 to present the medication information 210 to the user in the level of text complexity 218 that is determined based on the cognitive profile 214 of the user. The controller 108 may vary the medication information 210, based on different levels of text complexity 218, by selecting different words and phrases to describe the base concepts or content of the medication information 210. For a lower text complexity level, the controller 108 may filter out words and phrases that are determined to be too complex for the user. The controller 108 may supplement with smaller and/or more common words and phrases that the user would experience on a day-to-day basis.

In an example, the controller 108 controls the audio device 122 and/or the display device 116 to present the medication information 210 to the user by describing usage instructions for using the medication, descriptive information describing the medication, prohibitions/restrictions for taking the medication, and/or common side effects associated with the medication. The controller 108 may also provide a prompt to invite questions from the user if the user does not fully comprehend the information that was provided. For example, the controller 108 may formulate a question box that is displayed on the GUI 140. In another example, the controller 108 may control the audio device 122 to audibly ask the user if the user has any questions, followed by activating the microphone 124 to listen for any user-submitted question. Furthermore, the controller 108 may control the audio device 122 to ask more specific questions to obtain and update information about the user and whether the medication is appropriate for the patient. As examples, the controller 108 may control the audio device 122 and/or the GUI 140 to ask the user what other medications the patient is currently taking; whether the patient has any known allergies, such as specific drug allergies; and does the patient have any known adverse drug interactions?

The method optionally may include receiving a user voice command via a microphone 124, and analyzing the user voice command for content. The microphone 124 may convey electrical signals based on the user voice command to the controller 108, and the controller 108 analyze the electrical signals to determine the content of the user voice command. The controller 108 may then determine a response to the user voice command, and control the audio device 122 to respond to the user voice command using interactive voice response (IVR) technology. In an example, the controller 108 may control the audio device 122 to respond to the user voice command by audibly emitting information about one or more of side effects, adverse reactions, and/or food requirements associated with the medication.

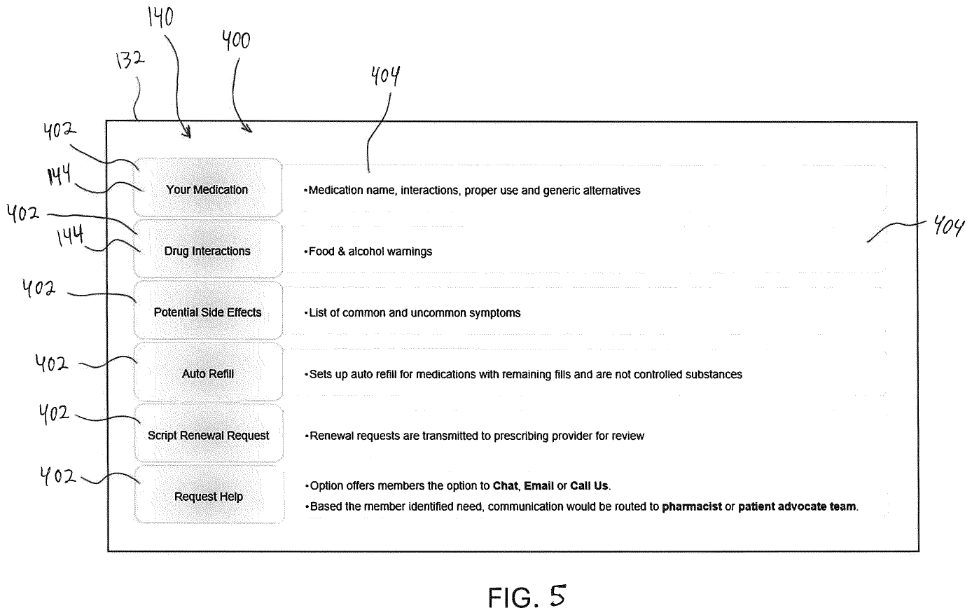

FIG. 5 illustrates an example menu 400 of module options 402 of the GUI 140 that is displayed on the display screen 132 of the MID system 100. The menu 400 lists multiple module options or modules 402. The module options or modules 402 may be user-selectable input elements 144 (e.g., virtual buttons that can be selected via touch, and/or may be provided as a braille-type mobile screen feature). The module options 402 may be interactive, closed caption modules that present additional information about the medication and usage to the user. Each module option 402 can be selected by the user to trigger the controller 108 of the MID system 100 to present the corresponding additional information. The module options 402 are directed to different categories of information about the medication. In the illustrated example, the module options 402 include “Your Medication,” “Drug Interactions,” “Potential Side Effects,” “Auto Refill,” “Script Renewal Request,” and “Request Help.” Next to each module option 402 is a respective summary 404 of the information that can be provided to the user by selecting that corresponding module option 402. For example, the summary 404 for the “Your Medication” module option 402 states that selecting that option 402 will yield information including “medication name, interactions, proper use, and generic alternatives.” The summary 404 for the “Request Help” module option 402 states that the option 402 “offers members the option to Chat, Email, or Call Us.” Additional module options 402 can include “Orders” (with the summary 404 displaying a current order status and summary of the medication if an order was placed) and/or “Medication Reminders” (with the summary 404 displaying a summary of medication adherence indicating the next dose reminder time).

In use, the user can select one of the module options 402 at a time by providing a voice command or touch input to a corresponding user input device 118. The MID system 100 described herein may be compatible with voice-recognition and computer voice generation technology so that the user can interact with the MID system 100 without needing to provide touch inputs and/or without needing to see the display screen 132. For example, the controller 108 may control the audio device(s) 122 to “read” the various module options 402 out loud to the user. In examples, the controller 108 may read the module options 402 and other information that is displayed out loud to the user in response to receiving a user voice command requesting the information be read out loud and/or in response to the cognitive profile 214 indicating that the user is blind, illiterate, has a reading disability, and/or the like. Upon selecting one of the module options 402, the controller 108 may modify the GUI 140 to present a new window or new text box that provides the additional information regarding the selected category. At the end of each module, the controller 108 may prompt the user, via the GUI 140 or the audio device(s) 122, what action the user would like to do next. The prompts may be provided with dictation, touch, key, and/or braille-type options.

In an embodiment, the MID system 100 accommodates several basic voice command functions to create an interactive user experience. For example, while the controller 108 is providing the medication information to the user according to the determined expression format 206, the user may have the option to provide voice commands that modify the rate, volume, and/or playback at which the medication information is presented to the user. Example voice command functions that the MID system 100 may accommodate include Rewind (e.g., jump back 15 seconds), Fast Forward (e.g., skip ahead 15 seconds), Replay, Stop, Pause, Volume Up, Volume Down, Slow (e.g., set playback speed to 0.5 or 0.75 speed), Speed Up (e.g., set playback speed to 1.25 or 1.5 speed), and/or the like. The user may be able to trigger these desired actions at any point while the user is viewing and/or listening to the medication information provided by the MID system 100. In another example, if the user would like additional information about a particular word or phrase in the medication information, the user may interject with a voice command requesting clarity. The user may state out loud, “Define [unfamiliar word]”. Alternatively, the user may provide a touch input to highlight a particular word or phrase and request that the controller 108 “Search Word.” By modifying the presentation of the medication information to the user according to user-selected functions, the MID system 100 creates an interactive user experience that enhances user comprehension.

Optionally, the method may include generating dosage reminders and controlling the audio device 122 and/or the display device 116 to provide the dosage reminders to the user at designated times. The controller 108 may receive a dosage schedule, and may track the dosage schedule via an internal clock. For each scheduled dosage time according to the schedule, the controller 108 may control the audio device 122 to emit an alert sound and/or may control the display device 116 to display a text-based notification on the screen 132. In another example, the controller 108 may control the communication device 114 to send a dosage reminder message to an external device, such as a phone of the user.

The methods described herein can be performed by execution of computer-readable instructions stored on a non-transitory computer-readable storage medium (e.g., random-access memory, flash memory, magnetic/optical storage, etc.) by the processor(s). In an example, the one or more processors 110 may store the input received from the user, which identifies the medication, in a first format in one or more network-based non-transitory storage devices (e.g., the memory 112 or the remote computing device 106). The storage device(s) have a collection of medication data stored thereon. The one or more processors 110 may provide remote access to the user's computing device 102 and at least one other user device over the network 104 so the user device 102 and/or the other user devices can provide updates to the input in the collection of medication data in real time. The user can provide updates through the GUI 140 provided via the mobile device 102. The user or the at least one other user may provide the updates to the input in a second format that differs from the first format. The second format may be dependent on the hardware and software platform used by the user or the at least one other user. A content server may convert the updates from the second format to the first format. The content server may then store the updates, in the first format, into the collection of medication data in the storage device(s). The one or more processors 110 may automatically generate a message containing the updates whenever the updates are stored. The one or more processors 110 may transmit the message to the user computing device 102 and the at least one other user device over the network 104 in real time so that each of the user device 102 and the at least one other user device has immediate access to up-to-date medication and patient information.

The MID system 100 addresses a specific problem rooted in mobile computing technology, namely that conventional mobile user interface frameworks lack mechanisms to dynamically adapt multimodal presentation of domain-specific information to a user's cognitive and accessibility constraints while maintaining legibility and synchronization in a live camera context. The controller 108 implements a real-time adaptive rendering pipeline that improves the functioning of the mobile computing device 102 by synchronizing AR overlays, captions, and narration to sensor-derived scene parameters and user profile signals, yielding lower latency, higher legibility under motion and low-light conditions, and reduced interaction error rates relative to static applications that merely display or speak preformatted label text.

In one example, the controller 108 performs pose estimation of the medication container in the live camera view by decoding the machine-readable code via the optical sensor 120, extracting fiducial features, and applying sensor fusion with inertial measurements from the mobile computing device 102 to determine a six-degree-of-freedom pose. The controller 108 registers the AR overlays to the container pose and applies perspective-correct transformations so that dosage, interactions, and warnings remain anchored to the physical item in view. This AR registration constitutes a specific improvement to mobile graphical rendering by stabilizing critical instructional overlays under device motion and camera jitter.

The controller 108 can use an adaptive readability engine that adjusts overlay parameters based on device and environmental inputs, including ambient illumination, motion blur signals, and contrast histograms computed from the camera feed of the optical sensor 120. In low-light conditions, the controller 108 can compute a luminance delta and automatically increases overlay brightness, edge thickness, and foreground/background contrast on the display device 116. When motion blur is detected, the controller 108 can reduce animation complexity and pins key instruction panels to the highest-stability regions of the frame to improve legibility. These operations can be performed via GPU-accelerated shaders and reduce rendering artifacts, thereby improving the mobile device's ability to present information under challenging conditions.

The controller 108 can reconcile device-level language and accessibility preferences with user-profile preferences by invoking operating system accessibility APIs, reading locale and assistive technology settings (e.g., screen reader state, caption preferences), and executing a deterministic rule engine to select final session parameters. The controller 108 can then configure the AR overlays, caption tracks, and IVR prompts to the selected language and accessibility profile, and ensures that all GUI 140 and overlay elements expose accessibility labels, roles, hint text, and focus navigation metadata required by screen readers such as TalkBack™ and VoiceOver™. This integration with OS-level services constitutes a technical improvement to the device's accessibility subsystem by enabling synchronized multimodal output across AR, audio, and captioning in real time.

The controller 108 can implement a content asset pipeline that stores AR assets, caption files, localized strings, and IVR prompts in canonical formats on the remote computing device 106 and synchronizes versioned packages to the mobile computing device 102 via the communication device 114. Upon synchronization, the controller 108 can transcode high-resolution three-dimensional animations to device-appropriate meshes or sprite sequences and converts caption files to platform-supported caption formats. The controller 108 can perform integrity checks, maintains version metadata, and caches assets in the memory 112 to reduce load times and network utilization during runtime. This pipeline provides a concrete improvement to mobile content delivery by decreasing latency, conserving bandwidth, and guaranteeing consistency of content versions.

The controller 108 may perform runtime synchronization between audio narration emitted by the audio device 122 and caption tracks rendered on the display device 116 by aligning phoneme timestamps, animation keyframes, and caption segment boundaries via a timebase controlled by the processor(s) 110. The controller 108 can detect drift and corrects caption timing with sub-frame adjustments to maintain legibility, thereby improving the accuracy and reliability of assistive output in mobile environments.

The controller 108 can use the cognitive profile 214 to configure non-trivial technical parameters of the rendering pipeline, including text complexity 218 selection, font metrics, line length, animation pacing, and icon emphasis weights. In one example, the controller 108 applies a rules-based simplification of technical terms and constructs caption segments with constrained sentence lengths and increased inter-segment spacing to support working-memory impairments. These transformations are computed programmatically and applied to AR overlays and IVR narration, thereby improving the device's ability to deliver information in forms that the user can process and retain.

When braille display compatibility is enabled, the controller 108 can map AR overlay content to braille output via operating system APIs and maintain coherence between visual overlays and tactile output by queuing braille segments aligned to overlay focus changes and narration timestamps. The controller 108 can validate braille segment boundaries and ensure that critical warnings and dosage instructions are surfaced with priority tactile cues to improve multi-sensory synchronization on the mobile computing device 102.

The MID system 100 can implement concurrent voice command control within AR mode by streaming audio from the microphone 124 to a voice recognition module while rendering overlays and narration. The controller 108 may prioritize low-latency commands such as “Pause,” “Rewind,” and “Define [word]” by reserving processor cycles and GPU resources for command handling queues and apply a concurrency model that prevents audio glitches and visual frame drops. This concurrency model can improve the functioning of the mobile computing device 102 by enabling real-time, interruption-safe control of multimodal output.

The arrangement and interplay of the above components and processes are not well-understood, routine, or conventional at least because conventional medication information applications present static text or audio without AR registration, do not perform sensor-driven adaptive readability adjustments, lack synchronized caption-narration alignment controlled by a hardware-accelerated timebase, and do not expose comprehensive accessibility metadata across AR overlays integrated with OS-level assistive technologies. The specific rendering pipeline, asset management, synchronization mechanisms, and accessibility integrations of the controller 108 materially improve the performance, reliability, and usability of the mobile computing device 102.

The operations described herein are necessarily performed with and tied to a particular machine comprising the mobile computing device 102 equipped with the optical sensor 120, audio device 122, display device 116, microphone 124, and/or processor(s) 110 executing the controller 108 and software application 146. The AR registration, sensor fusion, adaptive readability engine, synchronized captioning, and OS-level accessibility integrations depend on the capabilities of these components and cannot practically be performed as mere mental steps.

The MID system 100 integrates delivery of instructions with concrete, technical solutions that improve mobile device operation and human-computer interaction. By dynamically registering overlays to the physical medication container, adapting rendering to environmental conditions, reconciling device and user accessibility preferences, synchronizing multimodal outputs, and managing content via a versioned asset pipeline, the controller 108 applies the received information in a practical application and yields improvements to the computer's functionality rather than merely implementing the abstract idea on a generic computer.

The inventive subject matter described herein can use an artificial neural network (ANN) to perform the functions described herein. The use of specially trained ANNs to perform these operations realizes a number of improvements over traditional methods, as described herein. ANNs are a type of machine learning model used to perform a wide variety of complex tasks, including image recognition, speech recognition, pattern recognition, and detection of anomalies. An ANN can represent a biologically inspired algorithm that learns from training data. An ANN can be realized through software, hardware, or a combination of software and hardware. The structure of an example ANN has a series of layers, each comprising one or more neurons arranged in one or more neuron arrays. In one example, a neuron may comprise a register, a microprocessor, and at least one input. Each neuron produces an output, or activation, based on an activation function that uses the outputs of the previous layer and a set of weights as inputs. Each neuron in a neuron array may be connected to another neuron via a synaptic circuit. A synaptic circuit may include a memory for storing a synaptic weight. An example ANN may be a Deep Neural Network having an input layer, an output layer, and a plurality of fully connected hidden layers. ANNs are particularly useful in anomaly detection because they can effectively extract features in linear and nonlinear relationships. In some examples, an ANN may be implemented by an application-specific integrated circuit (ASIC). ASICs may be specially customized for a specific artificial intelligence application and provide superior computing capabilities and reduced electricity consumption compared to traditional CPUs.

The present specification can be used with the systems and methods described in U.S. Pat. No. 11,373,400, filed 18 Mar. 2019, titled METHODS AND SYSTEMS FOR IMAGE PROCESSING TO PRESENT DATA IN AUGMENTED REALITY, and granted 28 Jun. 2022, which is hereby incorporated by reference. For example, the glyph in the '400 patent can also operate as a machine-readable code. The support of the present description can include the container, the patient holding the container of the like. The medical information can include the refill information for the drug.

The present specification can be used with the systems and methods described in U.S. Pat. No. 10,127,729, filed 6 Apr. 2017, titled SYSTEM AND METHOD FOR FACILITATING VIRTUAL-SPACE-BASED PRESENTATION AND USER INTERACTION, and granted 13 Nov. 2018, which is hereby incorporated by reference.

The present specification can be used with the systems and methods described in U.S. Pat. No. 12,148,014, filed 15 May 2020, titled COMPUTERIZED AGGREGATION AND DISTRIBUTION ARCHITECTURE FOR DIGITAL HEALTH INFRASTRUCTURE, and granted 19 Nov. 2024, which is hereby incorporated by reference.

The above description is illustrative, and not restrictive. For example, the present description uses a printed label on a medical container as the illustrative embodiment. It is within the scope of the present invention to apply the methods and systems described herein to medical literature provided to patients or caregivers. Such literature can be provided by a hospital, a clinic, a provider, a home provider, a pharmacy or the like. An embodiment is directed to a computer-implemented method for dynamically providing medication information to a user, including: receiving, via an optical sensor of a mobile computing device, image data depicting a machine-readable code related to a patient; decoding the machine-readable code to identify a medical condition of the patient; obtaining, from a medical database stored in a first memory device, medical information related to the identified medical condition; determining, by one or more processors of the mobile computing device, a pose of the support for the machine-readable code in a live camera view rendered on a display device by performing sensor fusion of features extracted from the image data with inertial measurements of the mobile computing device; obtaining a user identifier that uniquely identifies the user and determining a cognitive profile of the user based on the user identifier by accessing a user database stored in one of the first memory device or a second memory device; obtaining device-level language and accessibility preferences via operating system accessibility application programming interfaces (APIs) of the mobile computing device and reconciling the device-level language and accessibility preferences with user-profile preferences stored in the user database; selecting, based on the cognitive profile and reconciled preferences, a first expression format comprising an overlay registered to the pose of the support with a text complexity level, synchronized audio narration, and closed captions in a selected language; initiating a presentation mode in response to decoding the machine-readable code and authenticating the user, and registering the overlay to the pose of the medical literature or the support of the machine readable code by applying perspective-correct transformations so that the overlay remains anchored to it; adapting, via an adaptive readability engine executed by the one or more processors, one or more overlay parameters based on environmental conditions sensed by the mobile computing device, including automatically increasing brightness and contrast in low-light conditions and reducing animation complexity upon detecting motion blur; and controlling the display device to present the overlay and controlling an audio device to emit synchronized narration and captions in the selected language.

For further example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments and are merely example embodiments. Other embodiments and modifications may be apparent to those of ordinary skill in the art upon reviewing the above description. As used herein, the terms “including” and “in which” are used as the plain-English equivalents of the respective terms “comprising” and “wherein.” Moreover, terms such as “first,” “second,” “third,” “upper,” “lower,” “bottom,” “top,” etc. are used merely as labels, and are not intended to impose numerical or positional requirements on their objects.

A processor or one or more processors as used herein can include hardware circuitry that includes and/or is connected with one or more computerized logic devices, such as one or more integrated circuits, application specific integrated circuits (ASICs), field programmable gate arrays, or the like.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

Use of phrases such as “one or more of . . . and,” “one or more of . . . or,” “at least one of . . . and,” and “at least one of . . . or” are meant to encompass including only a single one of the items used in connection with the phrase, at least one of each one of the items used in connection with the phrase, or multiple ones of any or each of the items used in connection with the phrase. For example, “one or more of A, B, and C,” “one or more of A, B, or C,” “at least one of A, B, and C,” and “at least one of A, B, or C” each can mean (1) at least one A, (2) at least one B, (3) at least one C, (4) at least one A and at least one B, (5) at least one A, at least one B, and at least one C, (6) at least one B and at least one C, or (7) at least one A and at least one C.

As used herein, an element or step recited in the singular and proceeded with the word “a” or “an” do not exclude the plural of said elements or operations, unless such exclusion is explicitly stated. Furthermore, references to “one embodiment” of the invention do not exclude the existence of additional embodiments that incorporate the recited features. Moreover, unless explicitly stated to the contrary, embodiments “comprising,” “comprises,” “including,” “includes,” “having,” or “has” an element or a plurality of elements having a particular property may include additional such elements not having that property. In the appended claims, the terms “including” and “in which” are used as the plain-English equivalents of the respective terms “comprising” and “wherein.” Moreover, in the following clauses, the terms “first,” “second,” and “third,” etc. are used merely as labels, and do not impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function devoid of further structure.

The above description is illustrative, and not restrictive. For example, the above-described embodiments (and/or aspects thereof) may be used in combination with each other. In addition, many modifications may be made to adapt a particular situation or material to the teachings of the subject matter without departing from its scope. While the dimensions and types of materials described herein define the parameters of the subject matter, they are exemplary embodiments. Other embodiments will be apparent to one of ordinary skill in the art upon reviewing the above description. The scope of the subject matter should, therefore, be determined with reference to the appended claims, along with the full scope of equivalents to which such clauses are entitled.

This written description uses examples to disclose several embodiments of the subject matter, including the best mode, and to enable one of ordinary skill in the art to practice the embodiments of subject matter, including making and using any devices or systems and performing any incorporated methods. The patentable scope of the subject matter is defined by the claims, and may include other examples that occur to one of ordinary skill in the art. Such other examples are intended to be within the scope of the claims if they have structural elements that do not differ from the literal language of the claims, or if they include equivalent structural elements with insubstantial differences from the literal languages of the claims.

What is claimed is:

1. A computer-implemented method for dynamically providing medication information to a user, the method comprising:
receiving, via an optical sensor of a mobile computing device, image data depicting a machine-readable code printed on a label of a medication container;
decoding the machine-readable code to identify a medication and obtaining, from a medication database stored in a first memory device, medication information about the medication that is identified;
determining, by one or more processors of the mobile computing device, a pose of the medication container in a live camera view rendered on a display device by performing sensor fusion of features extracted from the image data with inertial measurements of the mobile computing device;
obtaining a user identifier that uniquely identifies the user and determining a cognitive profile of the user based on the user identifier by accessing a user database stored in one of the first memory device or a second memory device;
obtaining device-level language and accessibility preferences via operating system accessibility application programming interfaces (APIs) of the mobile computing device and reconciling the device-level language and accessibility preferences with user-profile preferences stored in the user database;
selecting, based on the cognitive profile and reconciled preferences, a first expression format comprising an overlay registered to the pose of the medication container, a text complexity level, synchronized audio narration, and closed captions in a selected language;
initiating a presentation mode in response to decoding the machine-readable code and authenticating the user, and registering the overlay to the pose of the medication container by applying perspective-correct transformations so that the overlay remains anchored to the medication container under device motion;
adapting, via an adaptive readability engine executed by the one or more processors, one or more overlay parameters based on environmental conditions sensed by the mobile computing device, including automatically increasing brightness and contrast in low-light conditions and reducing animation complexity upon detecting motion blur; and
controlling the display device to present the overlay and controlling an audio device to emit synchronized narration and captions in the selected language.

2. The method of claim 1, wherein determining the pose of the medication container comprises extracting fiducial features proximate to the machine-readable code and performing six-degree-of-freedom pose estimation, and wherein registering the overlay comprises applying perspective-correct transformations that maintain overlay anchoring under camera jitter and device motion.

3. The method of claim 1, wherein selecting the text complexity level based on the cognitive profile comprises rules-based simplification of technical terms, constrained sentence lengths, and inter-segment spacing.

4. The method of claim 1, further comprising:
synchronizing caption timing by aligning phoneme timestamps of the narration and animation keyframes to a processor-controlled timebase; and
correcting caption drift with sub-frame adjustments to maintain legibility.

5. The method of claim 1, wherein reconciling device-level preferences with user-profile preferences comprises reading locale, screen reader state, caption preferences, and high-contrast mode via operating system accessibility APIs and applying a deterministic rule engine to select final session parameters.

6. The method of claim 1, further comprising:
exposing accessibility labels, roles, hint text, and focus navigation metadata for overlay elements and graphical user interface elements to enable screen readers to announce interactive content.

7. The method of claim 1, further comprising:
mapping the overlay to braille output via operating system APIs and maintaining coherence between visual overlay focus state and tactile output by queuing braille segments aligned to overlay focus changes and narration timestamps.

8. The method of claim 1, further comprising:
synchronizing versioned asset packages from a remote computing device and transcoding high-resolution animations to device-appropriate meshes or sprite sequences; and
caching assets locally in the second memory device to reduce latency and network utilization.

9. The method of claim 1, wherein adapting the overlay parameters comprises one or more of automatically increasing edge thickness, increasing foreground contrast, or increasing background contrast in low-light conditions.

10. The method of claim 1, wherein recognizing voice commands concurrently with AR rendering and narration is performed via an audio streaming pipeline that prioritizes low-latency command handling queues to prevent audio glitches and visual frame drops.

11. A system for dynamically providing medication information to a user via a computer device, the system comprising:
one or more memory devices that are non-transitory and computer-readable; and
one or more processors of a mobile computing device operably connected to an optical sensor, a display device, an audio device, and a microphone, the one or more processors configured to:
decode a machine-readable code captured by the optical sensor to identify a medication;
obtain medication information about the medication that is identified from a medication database stored in a first memory device and determine a pose of a medication container by fusing camera features with inertial measurements of the mobile computing device;
obtain a user identifier and determine a cognitive profile of the user by accessing a user database stored in one of the first memory device or a second memory device;
obtain device-level language and accessibility preferences via operating system accessibility APIs and reconcile the device-level language and accessibility preferences with user-profile preferences associated with the user identifier;
select an expression format comprising an overlay registered to the pose of a medication container, a text complexity level, synchronized closed captions, and audio narration aligned to a user language and cognitive profile; and
control the display device and audio device to output the overlay and narration with sub-frame caption synchronization,
the one or more processors further configured to adapt overlay parameters based on ambient illumination and motion blur, expose accessibility metadata on graphical user interface elements to enable screen readers and braille devices to announce interactive content.

12. The system of claim 11, wherein the one or more processors are configured to perform perspective-correct transformations to maintain overlay anchoring under camera jitter and device motion.

13. The system of claim 11, wherein the one or more processors synchronize caption timing by aligning phoneme timestamps and animation keyframes to a processor-controlled timebase and correct caption drift with sub-frame adjustments.

14. The system of claim 11, wherein the one or more processors expose accessibility labels, roles, hint text, and focus navigation metadata for overlay elements via operating system accessibility APIs to enable screen readers and braille devices.

15. The system of claim 11, wherein the one or more processors map the overlay to braille output via operating system APIs and maintain coherence between visual overlay focus state and tactile output.

16. The system of claim 11, wherein the one or more processors synchronize versioned asset packages from a remote computing device and transcode high-resolution animations to device-appropriate meshes or sprite sequences.

17. The system of claim 11, wherein the one or more processors recognize voice commands concurrently with AR rendering and narration using a concurrency model that prevents audio glitches and visual frame drops.

18. A non-transitory computer-readable medium storing instructions that, when executed by one or more processors of a mobile computing device operably connected to an optical sensor, a display device, an audio device, and a microphone, cause the mobile computing device to:
decode a machine-readable code captured by the optical sensor to identify a medication and determine a pose of a medication container by performing sensor fusion of camera features and inertial measurements;
obtain medication information from a medication database stored in a first memory device and obtain a user identifier to access a user database to determine a cognitive profile of a user;
obtain device-level language and accessibility preferences via operating system accessibility APIs and reconcile the device-level language and accessibility preferences with user-profile preferences to select an expression format comprising an overlay registered to the pose of the medication container, a text complexity level, synchronized closed captions, and audio narration in a selected language;
adapt overlay parameters via an adaptive readability engine based on ambient illumination and motion blur; and
control the display device and audio device to output the overlay and synchronized narration and captions, while recognizing voice commands to control playback of the overlay and narration.

19. The non-transitory computer-readable medium of claim 18, wherein the instructions further cause the mobile computing device to expose accessibility metadata on the overlay and graphical user interface elements to enable screen readers and braille devices to announce interactive content.

20. The non-transitory computer-readable medium of claim 18, wherein the instructions further cause the mobile computing device to synchronize caption timing by aligning phoneme timestamps and animation keyframes and to correct caption drift with sub-frame adjustments.
